(19) United States
(12) Patent Application Publication
(10) Pub. No.: US 2013/0338620 A1
(43) Pub. Date: Dec. 19, 2013

(54) Absorbent article with a bacterial isolation pad and hygenic gripping tabs

(71) Applicant: Jane Speak, Tequesta, FL (US)

(72) Inventor: Jane Speak, Tequesta, FL (US)

(21) Appl. No.: 13/495,624

(22) Filed: Jun. 13, 2012

(51) Int. Cl. A61F 13/505; A61F 13/511

(52) U.S. Cl. 604/378

(57) ABSTRACT

An absorbent article for use as an incontinence pad or pantiliner includes a bacterial isolation pad which overlays the rear section of a body-side cover to block bacteria in the anal region from contacting the urine-receiving portion of the absorbent article. A liquid impermeable gripping tab is attached at each end of the absorbent article for ease of handling and to provide hygienic removal of the article.

A portion of the disclosure of this patent document contains material which is subject to copyright protection. The copyright owner has no objection to the facsimile reproduction by anyone of the patent document of the patent disclosure, as it appears in the Patent and Trademark Office patent file or records, but otherwise reserves all copyright rights whatsoever.

### FIELD OF THE INVENTION

The present invention relates generally to an improvement to a personal hygiene absorbent article used as an incontinence pad, pantiliner, diaper, or other absorbent undergarment. More specifically, the present invention pertains to absorbent articles designed to absorb urinary liquids and limit bacterial contamination from the anus of an individual to the urethra and also to provide hygienic grips for secure and sanitary handling of a soiled article.

### BACKGROUND OF THE INVENTION

Urinary tract infections (UTIs) are the second most common type of infection in the body, most of which are caused by bacteria that live in the bowel. Women are especially susceptible because of the short distance bacteria have to travel from the anus to the urethra. Fecal drainage, improper cleansing after evacuation and extended wear of a wet absorbent pad or undergarment are all considered risk factors for a UTI. A Norwegian study titled, “Pad per day usage, urinary incontinence and urinary tract infections in nursing home residents,” published online in July 2010 on behalf of the British Geriatrics Society, concluded that the use of absorbent pads is associated with an increased risk of developing UTIs.

Women with urinary incontinence rely on absorbent pads, pantiliners and undergarments, which are readily available and simple to use. These absorbent articles use a continuous absorbent core extending to the front and rear from the point of urinary insult, allowing a larger area for absorption of urine, but also providing a path for migration of anal bacteria to the urethra along a dampened topsheet or urine-soaked absorbent core.

### DESCRIPTION OF THE PRIOR ART

Omli, Ragnhild et al., “Pad per day usage, urinary incontinence and urinary tract infections in nursing home residents,” published online on Jul. 14, 2010 by Oxford University Press on behalf of the British Geriatrics Society in ageing.oxfordjournals.org. This article discusses the results of a study performed in six nursing homes. The study concluded that the residents of nursing homes who used absorbent pads were at an increased risk of developing urinary tract infections (UTIs).

U.S. Pat. No. 7,943,158 discloses a means to produce antimicrobial activity in fabric material. The means reduces the microbial content on an article in close proximity to an animal body by providing molecular iodine to the stabilized reagents when at least two reactants are activated by aqueous and/or alcohol materials. Use in a diaper is mentioned, but no other design changes are disclosed.

In response to concerns of bacterial contamination, barrier webs impermeable or toxic to microorganisms disclosed in U.S. Pat. No. 6,083,602 and fibers which trap bacteria disclosed in U.S. Pat. No. 7,947,292 have been created for use in absorbent hygiene products such as diapers, napkins, and tissues. Use of the fabrics in diapers and other absorbent articles is mentioned, but no other design changes are disclosed.

U.S. Pat. No. 6,083,602 discloses a web material which has been treated with a curable shear thinned thixotropic polymer composition. The material is then substantially impermeable to liquids, permeable to gases, and impermeable to microorganisms.

U.S. Pat. No. 7,947,292 discloses a hygiene product containing fibrous material for removing or inactivating microbes. This is accomplished by the fibrous material containing functional groups capable of interacting with bacteria cell walls.

From these three patents, it appears that the only change foreseen to existing absorbent articles is use of their fabric in existing designs or addition of a fabric covering. How these bacteria control fabrics are incorporated into commonly used incontinence products in the general population, where cost, comfort, and performance are major factors, remains to be seen.

Some absorbent articles, such as U.S. Pat. No. 5,755,710 and U.S. Published Patent Application No. 2003/0093045, have a defined urine strike window, intended to provide leakage protection. This feature does not limit migration of anal bacteria as fecal drainage trickles along the liquid impervious topsheet down into the strike window. To avoid this occurrence, someone skilled in the art may add an entry zone for fecal insult in the anal region of the article. Unfortunately, this access then establishes a direct path for migration of anal bacteria to the urethra through the continuous absorbent core when it becomes saturated with urine, as in a regular incontinence pad.

U.S. Pat. No. 5,755,710 discloses an absorbent article for use in the perineal area of the body. The article has an absorbent core which has an upper body facing surface, a lower garment facing surface, first and second longitudinal sides, and first and second transverse ends. A body fluid impervious sealing wrapper having upper and lower portions each having longitudinally and transversely extending edges along which the portions are joined is formed around the absorbent core so as to enclose at least the entirety of the garment facing surface, the longitudinal sides, and the transverse ends, thereby forming a seal. The article has a centrally disposed opening in the upper portion of the sealing wrapper thus forming a window on the body facing surface of the absorbent core that exposes it to fluid flow.

U.S. Published Patent Application No. 2003/0093045 discloses an absorbent article which provides leakage protection and protection for the skin of the individual using the article. The article includes a selectively-permeable top sheet, a substantially impermeable back sheet, and an absorbent core located between the top sheet and the back sheet. The top sheet has a treated hydrophilic zone and an untreated hydrophobic zone.

Sanitary handling of urine-soaked incontinence pads is also a concern. Today's commonly used incontinence pads are designed to be gripped by the peripheral fringes for removal. While the back sheet of the pad is liquid impervious, the liquid pervious top sheet on the top side of the fringe allows urine to seep onto the top side of the fringe.

U.S. Pat. No. 6,595,977 provides a tape tab, which extends from the sides or ends of an absorbent article to provide a hygienic means of removing the article. Since the primary function of the tape tab is to adhesively maintain the position of an absorbent article, all claims are specifically for an adhesive tape tab. This design not only requires the user to detach the tab from the user's garment before removal, but also extends the gripping point away from the body of the absorbent article, contributing unwanted movement to a urine-soaked article during removal and disposal.

U.S. Pat. No. 7,553,303 is the applicant's prior patent which discloses an undergarment having at least one aperture in the crotch region for the removal of and insertion of absorbent pads.

What is lacking in the art is an absorbent article for an incontinent person that physically separates anal bacteria from urine absorbed by the article, without reducing the absorption area of the article or requiring the use of special fibers, barrier webs, or other costly or special manufacturing processes. Also lacking in the art are gripping points to allow quick and easy hygienic removal of a fully saturated incontinence pad.

### SUMMARY OF THE INVENTION

An absorbent article for use as an incontinence pad or pantiliner includes a bacterial isolation pad which overlays the rear section of a body-side cover to block bacteria in the anal region from contacting the urine-receiving portion of the absorbent article. A liquid impermeable gripping tab is attached at each end of the absorbent article for ease of handling and to provide hygienic removal of the article.

In a second embodiment of the present invention, the incontinence pad of the first embodiment includes a removably attached bacterial isolation pad. A gripping tab at the rear-facing edge of the isolation pad facilitates removal of the isolation pad. Attachment of the isolation pad to the incontinence pad is guided by a colored or embossed placement indicator on the body-side cover.

In a third embodiment of the present invention, the absorbent article can be used as an absorbent undergarment, a diaper, or a pull-up with a bacterial isolation pad attached to the anal region of the undergarment, the diaper, or the pull-up.

In a fourth embodiment of the present invention, the absorbent undergarment of the third embodiment is provided with a removably attached bacterial isolation pad. A gripping tab at the rear-facing edge of the isolation pad facilitates removal of the isolation pad. Attachment of the isolation pad to the absorbent undergarment is guided by a colored or embossed placement indicator on the body-side cover of the garment.

Accordingly, it is an objective of the present invention to provide an absorbent article for use as an incontinence pad, pantiliner, or diaper-type undergarment. A bacterial isolation pad is secured to and overlays a rear section of the incontinence pad on the side of the incontinence pad which faces a user.

It is a further objective of the present invention to prevent bacteria from the anal region of an individual from coming in contact with urine and causing urinary tract infections.

It is yet another objective of the present invention to provide an absorbent article for use as an incontinence pad or pantiliner which includes a gripping tab secured to a bacterial isolation pad to assist in removal of the absorbent article from a panty or other undergarment.

It is a still further objective of the present invention to provide an absorbent article for use as an incontinence pad or pantiliner which includes a gripping tab secured to a bacterial isolation pad to provide hygienic removal and disposal of the isolation pad.

It is a still further objective of the present invention to provide an absorbent article for use as an incontinence pad or pantiliner. A bacterial isolation pad is secured to and overlays a rear section of the incontinence pad on the side of the incontinence pad which faces a user. The incontinence pad also includes an indicator such as indicia to assist a user in the correct placement of the bacterial isolation pad onto the incontinence pad.

It is a still further objective of the present invention to provide an undergarment, a diaper, or a pull-up with a bacterial isolation pad which prevents bacteria from the anal region of an individual from coming in contact with urine from the same individual.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a top view of the body-facing side of an embodiment of the present invention used as an incontinence pad;

FIG. 2 is a longitudinal cross-section of the embodiment of FIG. 1, with edges not shown bonded;

FIG. 3 is a top view of a removable bacterial isolation pad of the present invention;

FIG. 4 is a longitudinal cross-section of the removable bacterial isolation pad shown in FIG. 3;

FIG. 5 is a partial section of the rear portion of the body-facing side of an embodiment used as an incontinence pad with a placement indicator for a removable bacterial isolation pad; and

FIG. 6 is a front view of an embodiment of the present invention used as an absorbent undergarment revealing an attached bacterial isolation pad in the anal region of the interior body-side cover of the garment.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

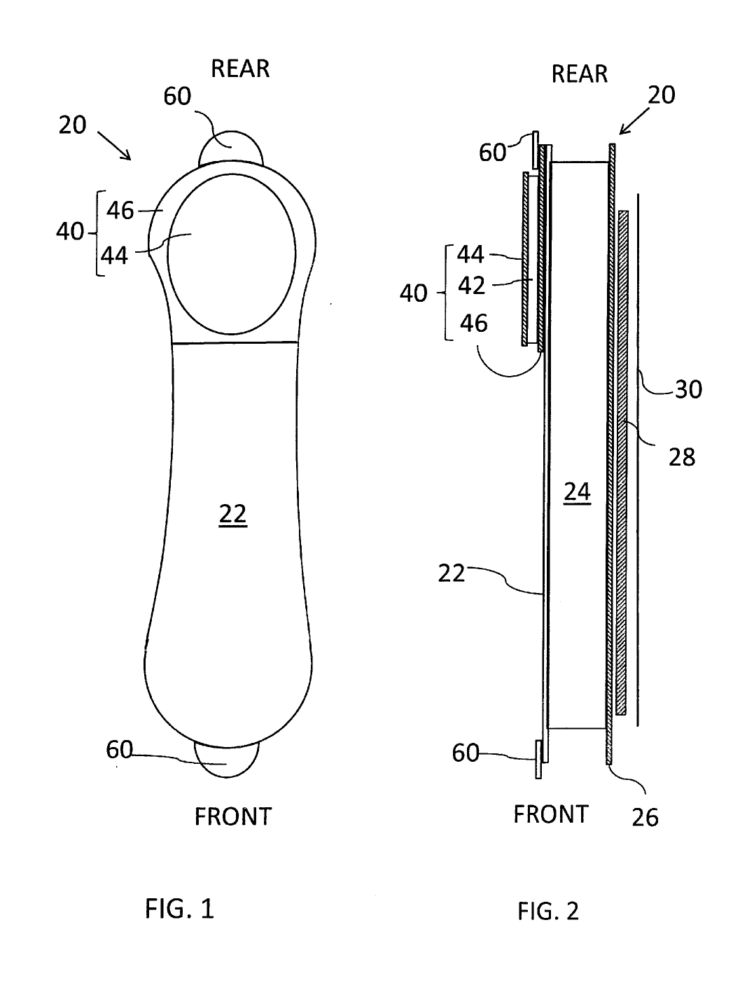

FIGS. 1-6, which are now referenced, illustrate the present invention and the manner in which it is assembled. A preferred embodiment of the present invention is illustrated in FIGS. 1 and 2. In this embodiment, an absorbent article 20 is illustrated. In this preferred embodiment, the absorbent article is an incontinence pad 32. The incontinence pad is used by an individual whenever there is a chance that bodily fluids and/or wastes can or will be discharged unexpectedly or at an inopportune time. One of the main uses or purposes of an incontinence pad is to absorbed and retain fluids discharged from an individual so that the fluids cannot be passed onto clothing or other materials which would result in an unsightly stain or damage of the clothing. The incontinence pad 32 includes an absorbent core 24, FIG. 2 which absorbs fluids. A permeable body-side cover or layer 22 is secured to an upper surface of the core 24, as illustrated in FIG. 2. The upper surface defined as the surface of the core 24 which is closest to the body of an individual when the pad 32 is in use. The permeable body-side cover 22 is formed from a fluid permeable material. In the preferred embodiment, urine from an individual passes through the body-side cover 22 and is absorbed into the core 24. The core 24 is made from a material which will absorb and retain fluids. Thus, the urine is retained in the core 24 so that the urine will not move or be transported onto an individual's clothing or other areas of an individual's body. This prevents wetting and unsightly stains on garments.

Secured to the lower surface or underside of the core 24 is liner or fluid impermeable layer 26. In a preferred embodiment, the liner 26 is a garment side liner. The fluid impermeable layer 26 is constructed and arranged to prevent any fluids from passing from the core 24 onto a garment or article of clothing, (not shown). The fluid impermeable layer 26 has an adhesive strip 28 secured to a first side, as illustrated in FIG. 2. The adhesive strip 28 is employed to both secure the incontinence pad to a garment or an article of clothing and to prevent the incontinence pad from moving when in use. Normally the incontinence pad 32 is secured to an undergarment such as panties. As a result of an individual moving, walking, sitting, etc. the panties will move relative to the individual's body. If the incontinence pad 32 were not secured to the panties, it would be moved out of its desired or preferred position as a result of the individual's movement. The desired position of the incontinence pad 32 is adjacent the lower portion of an individual's body where fluids are released or expelled, the vagina and the anus. In a preferred embodiment, the incontinence pad 32 has a narrow central portion located between the wider rounded distal end portions. The dimensions of this embodiment of the incontinence pad 32 are 10 inches long, along a centerline of the pad, 3 inches wide, at the widest portion of the pad, and 2.5 inches wide, at the narrowest or central portion of pad 32.

A bacterial isolation pad 40 is secured to one end of the incontinence pad 32, as illustrated in FIGS. 1 and 2. The end at which the bacterial isolation pad 40 is secured is considered to be the rear or back end of the incontinence pad 32. When the incontinence pad 32 is placed against an individual's body the rear end of the incontinence pad is located at or toward the rear side of the individual's body. The ideal position of the bacterial isolation pad 40 is adjacent an individual's anus. The bacterial isolation pad 40 includes a fluid absorbent layer or pad 42 which is from a material which will absorb and retain fluids. A fluid permeable topsheet or layer 44 is secured to a first side of said bacterial isolation pad 40. Fluids discharged from an individual's anus pass through the topsheet 44 and into pad 42 where they are retained. A fluid impermeable backsheet or layer 46 is secured to a second side of said layer or pad 42. The second side of the pad 42 is opposite the first side of the pad 42, as illustrated in FIG. 2 and FIG. 4. Preferably, the topsheet 44 is in contact with an individual's anal region of their body.

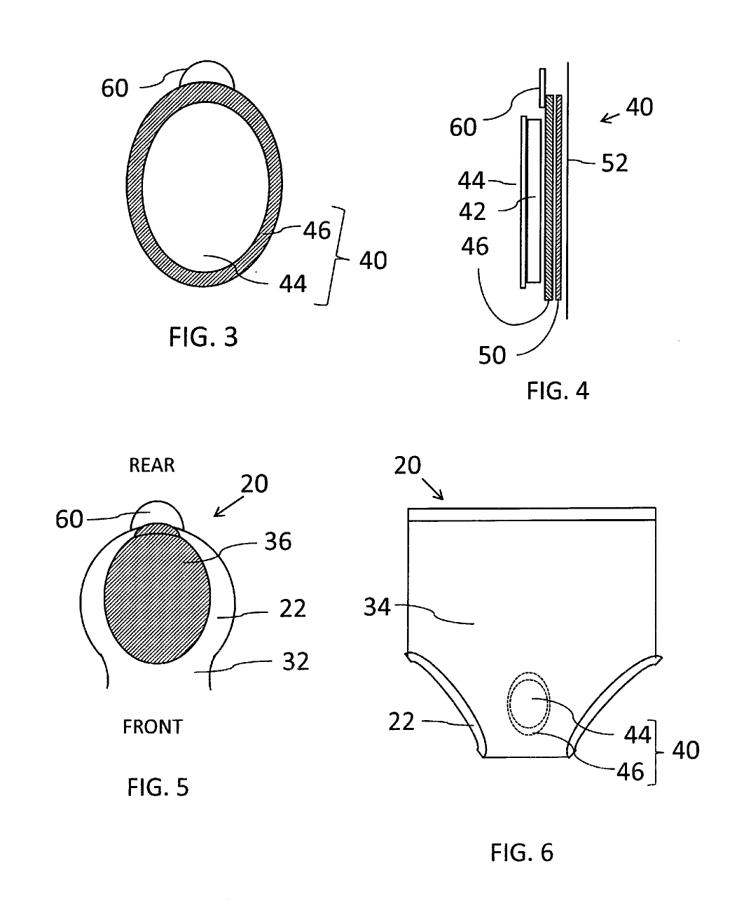

As illustrated in FIGS. 1 and 3, the bacterial isolation pad 40 is oval shaped. However, any other shape may be employed. The only criteria is that the bacterial isolation pad 40 trap and retain fluids discharged from the anus of an individual. This prevents these fluids from coming into contact with urine and/or other fluids discharged from an individual. Intermixing of anal fluids and urine may result in a urinary tract infection. The present invention prevents this. The outer edges of topsheet 44 and backsheet 46 preferably extend beyond the outer edges of fluid absorbent layer 42, as illustrated in FIG. 2 and FIG. 4. The outer edges of topsheet 44 and backsheet 46 extend at least 0.25 inches beyond the outer edges of fluid absorbent layer 42, in a preferred embodiment. The front-facing edge and downward surface of the backsheet 46 float freely over the body side cover 22 of pad 32.

Gripping tab 60 is secured to at least one end of the pad 32, as illustrated in FIGS. 1 and 2. The gripping tab(s) 60 provide hygienic grips for secure and sanitary handling of the incontinence pad 32. The gripping tabs 60 are made from a liquid impervious material in order to provide a firm gripping surface for an individual removing a soiled incontinence pad. In a preferred embodiment, the gripping tabs 60 are arched shaped. They are 0.75 inches high and 1.5 inches at their widest point. The tabs 60 are preferably secured at the center portions of the ends of the incontinence pad 32.

The fluid impermeable layer 26 has one or more tracks of releasable adhesive 28 for releasably securing the pad 32 to a garment. The releasable adhesive 28 is secured on a first side to the fluid impermeable layer 26. A removable adhesive peel strip 30 is secured to a second side of the releasable adhesive 28. The peel strip 30 is designed to be removed by an individual just prior to positioning and securing the incontinence pad 32 to the crotch region of an undergarment or other garment. The peel strip 30 is generally slightly larger than the overall dimension of the adhesive 28 for manufacturing purposes and also for ease of removal by an individual. In a preferred embodiment the adhesive 28 is a releasable garment adhesive. Additionally other releasable securing means and configurations of attachment can also be employed to releasable secure the incontinence pad 32 to a garment. The peel strip 30 is preferably paper or plastic, coated on one side for ease of release from the adhesive 28. Additionally, other material can be used for the peel strip 30.

The body-side cover 22 and fluid impermeable layer 26 can be made from materials commonly used in the industry to produce fluid permeable or fluid impermeable materials. Additionally, these elements can be made from a material and treated or coated with material(s) to render them fluid permeable or fluid impermeable. Woven or non-woven materials formed from natural or synthetic fibers and/or other materials can be employed to make these elements. Fluid impermeable layer 26 can be made from fluid impermeable materials such as, but not limited to, polyaminates, polymeric films such as polyethylene, polypropylene, polyester, cellophane, etc. or a bi-component film. Non-woven fabrics, impregnated fluid repellent paper, and other materials can also be used. The outer edges of the body-side cover 22 and the garment side liner 26 are sealed together to retain the fluid absorbent core 24. These edges can be joined by pressure sensitive adhesives, heat sensitive adhesives, ultrasonics, or other known joining applications which prevent fluid discharge beyond these outer edges. The sealing of these edges may be continuous, discontinuous, or staggered.

Absorbent core 24 is illustrated as being formed from a single absorbent layer. However, multiple absorbent and/or wicking layers can be employed to form the absorbent core 24. The materials from which the absorbent core 24 is made will determine the fluid capacity of the core 24. Pulp, super-absorbent polymers, and other organic and inorganic materials well known in the art are illustrative of materials which are commonly employed for their absorbent properties.

In a preferred embodiment of the present invention, the bacterial isolation pad 40 is secured to the outer periphery of the incontinence pad 32 of the overlay areas. It can also be directly bonded to the body side cover 22 directly below or attached in various configurations or locations. The size, shape, construction, and configuration of the bacterial isolation pad 40 may be varied to accommodate various absorbency, placement, and design criteria.

The topsheet 44 and the fluid absorbent core 24 can cover a larger or smaller area of the liquid impermeable backsheet 46. The topsheet 44 may be made from the same material as those used for the body-side cover 22, described herein above. It can also be made from a treated fabric like the fabric described in U.S. Pat. No. 7,943,158. Microorganism barrier webs, like those described in U.S. Pat. No. 6,083,602 or bacteria trapping fibrous materials of the type described in U.S. Pat. No. 7,947,292 can also be used. Further, an absorbent layer 42 of the bacterial isolation pad 40 may be of the same materials as the absorbent core 24 of the incontinence pad 32 or one or more sheets of a bacteria containment product, as described for the topsheet 44.

The backsheet 46 of the bacterial isolation pad 40 can be made from the same materials as the garment side liner 26, described herein above. However, texture and comfort must also be considered because of the extended contact between the backsheet 46 and the body of an individual. One or more layers of bacteria trapping fibrous materials and/or microorganism barrier webs can be used as a combination backsheet 46, absorbent core 42, and topsheet 44, with a liquid impervious incontinence pad 32 interface.

A second embodiment of the present invention is disclosed wherein the absorbent article 20 of the previously described embodiment illustrated in FIGS. 1 and 2 is an incontinence pad 32 with a removably attached bacterial isolation pad 40 as illustrated in FIGS. 3 and 4. The bacterial isolation pad 40 of the second embodiment is the same as the one previously disclosed, except for the means and method of securing the body-side cover 22 to the incontinence pad 32. In the second embodiment, the backsheet 46 of a bacterial isolation pad has one or more tracks of releasable liquid resistant garment adhesive 50 coupled with an adhesive peel strip 52. This construction permits releasable securement of the bacterial isolation pad 40 onto the incontinence pad 32. A placement indicator 36, FIG. 5, is placed on the incontinence pad 32 to assist in the proper placement of the bacterial isolation pad 40. The adhesive materials used for the adhesive 50 in this embodiment are the same as those used in the prior embodiment, except that a liquid resistant means of adhesion is needed to assure securement of the bacterial isolation pad 40 when the body-side cover 22 is wet. The area of adhesion of the bacterial isolation pad 40 may be along the outer perimeter of the bacterial isolation pad 40 or in various other configurations to assure proper securement of the pad 40 to the body-side cover 22.

An arched shaped gripping tab 60 is secured at the center of the rear-facing end of the bacterial isolation pad 40. The gripping tab 60 can be secured at various other positions on pad 40. A preferred embodiment of the gripping tab 60 is 0.5 inches high and 1.0 inches wide at its widest point. The gripping tab 60 is lifted to remove the bacterial isolation pad 40. Tab 60 may be gripped together with the gripping tab 60 of the incontinence pad 32, which it partially overlays, thereby lifting the entire article 20. A placement indicator 36 on the body-side cover 22, FIG. 5, illustrates the base portion of a gripping tab 60 for the bacterial isolation pad 40 but not its full size. Also indicated in FIG. 5, when a removable bacterial isolation pad 40 is used, the rear-facing gripping tab 60 for an incontinence pad 32 is then directly secured to the body-side cover 22, like the front-facing tab 60 as illustrated in FIGS. 1 and 2.

The absorbent layer 42 and topsheet 44 of the bacterial isolation pad 40 can be the same size as that of the first embodiment with a backsheet 46 which has a body-facing perimeter border of 0.12 inches in a preferred embodiment. The size, shape, configuration, and construction of the components of the present invention may be varied to meet various absorbency, placement, and design criteria.

A third embodiment of the present invention is illustrated in FIG. 6, wherein an absorbent article 20 is an absorbent garment 34. A bacterial isolation pad 40 is secured to the anal region of the garment 34, which is an undergarment in this embodiment. The garment 34 can also be a diaper or a pull-up undergarment. Garment 34 includes a circular waist opening and a pair of leg openings. The garment 34 also includes a front panel and a rear panel, with a crotch region therebetween.

The fluid absorbent layer or pad 42 and topsheet 44 of the bacterial isolation pad 40 are the same size as the pad 40 of the first embodiment. The backsheet 46 preferably has a body-facing perimeter of 0.12 inches. Materials and joining methods for these elements have been described in the first embodiment of the present invention. The size, shape, configuration, and construction of these components may be varied to meet various absorbency, placement, and design criteria.

A fourth embodiment of the present invention is illustrated in FIG. 6 wherein an absorbent article is a garment 34 with a removably secured bacterial isolation pad 40. The bacterial isolation pad 40 of this fourth embodiment is the same as that illustrated in FIGS. 3 and 4 herein above and a gripping tab 60 is provided to assist in removing the bacterial isolation pad 40 from the garment 34. The garment 34 can be made from any material which is suitable for use as a diaper-type garment or a reusable, washable garment.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An article for protection against anal contamination comprising:
an absorbent core;
a permeable layer;
a fluid impermeable liner;
said absorbent core positioned between said permeable layer and said fluid impermeable liner, said permeable layer and said fluid impermeable liner together completely enclosing said absorbent core;
a pad secured to one end of said permeable layer, said pad being substantially smaller than said absorbent core;
said article being adapted to be positioned adjacent and covering at least a portion of a body in an anal region thereof;
said pad being positioned in a region to absorb fluids discharged from an anus and prevent said discharged fluids from contacting other fluids discharged from a body.

2. The article of claim 1 wherein said pad is formed of a material which will absorb and retain fluids;
a fluid permeable topsheet secured to a first side of said pad;
a fluid impermeable backsheet secured to a second side of said pad, said second side of said pad being on an opposite side of said pad from said first side of said pad.

3. The article of claim 1 including at least one first tab secured to at least one end of said article.

4. The article of claim 3 wherein said at least one first tab is secured to at least one end of said permeable layer.

5. The article of claim 1 further including an adhesive strip secured on a first side to said fluid impermeable layer opposite said absorbent core, for removably securing said article to an article of clothing.

6. The article of claim 5 including a peel strip secured to a second side of said adhesive strip, said peel strip constructed and arranged to prevent adhesive on said adhesive strip from contacting an object prior to said article being secured in a desired location.

7. The article of claim 2 including at least one track of releasable adhesive secured to said fluid impermeable backsheet on a side of said fluid impermeable backsheet opposite said pad.

8. The article of claim 7 including at least one second tab secured to said fluid impermeable backsheet.

9. An article of clothing for protection against anal soiling comprising:
a garment;
an absorbent core;
a permeable layer;
a fluid impermeable liner;
said absorbent core positioned between said permeable layer and said fluid impermeable liner, said permeable layer and said fluid impermeable liner together completely enclosing said absorbent core;
a pad secured to one end of said permeable layer, said pad being substantially smaller than said absorbent core;
said pad being adapted to be positioned adjacent and covering at least a portion of a body in an anal region thereof;
said pad being secured to said garment and positioned in a region to absorb fluids discharged from an anus and prevent said discharged fluids from contacting other fluids discharged from a body.

10. The article of clothing of claim 9 wherein said pad is formed of a material which will absorb and retain fluids;
a fluid permeable topsheet secured to a first side of said pad;
a fluid impermeable backsheet secured to a second side of said pad, said second side of said pad being on an opposite side of said pad from said first side of said pad.

11. The article of clothing of claim 10 including at least one track of releasable adhesive secured to said fluid impermeable backsheet on a side of said fluid impermeable backsheet opposite said pad.

12. The article of clothing of claim 11 including a peel strip secured to said at least one track of releasable adhesive, said peel strip constructed and arranged to prevent adhesive on said adhesive strip from contacting an object prior to said article being secured in a desired location.

13. The article of clothing of claim 10 including at least one tab secured to said fluid impermeable backsheet.

14. An article of clothing for protection against anal soiling comprising;
a pad secured to said article of clothing, said pad is formed of a material which will absorb and retain fluids;
a fluid permeable topsheet secured to a first side of said pad;
a fluid impermeable backsheet secured to a second side of said pad, said second side of said pad being on an opposite side of said pad from said first side of said pad.
